(19) United States
(12) Patent Application Publication
(10) Pub. No.: US 2020/0397615 A1
(43) Pub. Date: Dec. 24, 2020

(54) Welding Hood

(71) Applicant: Richard Nelson Williams, Washington, PA (US)

(72) Inventor: Richard Nelson Williams, Washington, PA (US)

(21) Appl. No.: 16/906,283

(22) Filed: Jun. 19, 2020

Related U.S. Application Data
(60) Provisional application No. 62/864,428, filed on Jun. 20, 2019.

(51) Int. Cl. A61F 9/06

(52) U.S. Cl. CPC A61F 9/065; A61F 9/06

(57) ABSTRACT

A welding hood for protecting an operator's face comprising a face protective shield having a concave rearward side for receiving the operator's face, a viewing opening extending through the shield, wherein the viewing opening is located in a position in front of the eyes of the operator while the hood is fixed to the operator's head. The welding hood includes an eye box located adjacent the viewing opening. The eye box has walls framing the viewing opening and extend inwardly from the rearward side of the shield. The walls are configured for blocking reflective arc flashes and/or light from entering the operator's field of view during welding. The welding hood has sidewalls of varying width and a neck attachment for added protection. The welding hood also includes a lens located within the viewing opening and at least one strap for securing the welding hood to the operator's head.

### CROSS REFERENCE TO RELATED APPLICATIONS

This application claims priority to U.S. Provisional Patent Application Ser. No. 62/864,428, entitled “SPARCMASTER Welding Hood”, filed Jun. 20, 2019, which is hereby incorporated by reference in its entirety.

### BACKGROUND OF THE INVENTION

### Field of the Invention

The present invention is directed to a welding hood, and more particularly, to a welding hood having a fitted eye box and extended side portions, providing improved safety and comfort.

### Description of Related Art

Welding helmets or hoods are well-known protective gear for welders and have been in use since around the 1930's. One example of a currently available and known welding hood is shown in prior art FIGS. 1-3 of the present application. As can be seen from the prior art figures, many of these known models provide little comfort to the operator, provide little to no blockage of reflective arc flash, sunlight, and/or shop-lighting from entering behind the face shield, and provide little to no blockage of sparks, dirt, and/or debris from getting in the eyes of the operator. These known welding hoods also do not provide additional protection of the operator's ears and/or neck, leaving much of these areas exposed.

The welding hood of the present invention overcomes the deficiencies of the prior art by providing numerous safety features for the eyes, ears, and neck of the operator while also increasing the comfort to the operator.

### SUMMARY OF THE INVENTION

In accordance with one aspect, the present disclosure is directed to a welding hood for protecting an operator's face comprising a face protective shield having a front or forward side, which is typically convex in shape, and a back or rearward side, which is typically concave in shape and configured for receiving the operator's face, a viewing opening extending through the shield, wherein the viewing opening is located in a position in front of the eyes of the operator while the hood is fixed to the operator's head. The welding hood also includes an eye box located adjacent the viewing opening. The eye box has at least one wall framing at least a portion of the viewing opening and extends inwardly from the rearward side of the shield. According to one embodiment, a plurality of walls are provided for fully framing or completely surrounding the viewing opening so as to block at least one of reflective arc flashes, sunlight, and shop-lighting from entering the operator's field of view during welding. The welding hood also includes a lens located within the viewing opening for protecting the operator's eyes while allowing the operator to look through the viewing opening and at least one strap for securing the welding hood to the operator's head.

The walls of the eye box can be contoured to fit to the operator's face surrounding the operator's eyes. In particular, the walls can be contoured or can be formed from a flexible material, such as rubber or a foam material, so that the walls fit flush against the operator's face to reduce the amount of sparks, dirt, and/or debris from coming into contact with the operator's eyes.

The lens can comprise a light-filtering lens for filtering light passing through the opening toward the rearward side of the hood. The welding hood can include a holding member for holding the lens, wherein the holding member is capable of pivoting between an open and closed position about the viewing opening. The provision of a pivoting holding member enables the operator to lift the eye lens for better viewing of a fit-up or grind, if needed, or for communication with another person, without having to lift the protective shield from their face. This provides an added safety measure to the welding hood. The holding member can be configured for removably holding the lens so that the lens can be replaced.

According to one embodiment, the at least one strap for securing the welding hood to the operator's head can comprise an elastic strap that wraps around the operator's head. It can be appreciated that the strap can be formed from other known materials. The strap can be adjustable so that the welding hood is firmly secured to the operator's head.

The face protective shield can include a first side portion having a first width configured for extending over at least a portion or completely over the ear of the operator and a second side portion having a second width. According to one embodiment, this second width can be the same as the first width so as to provide added coverage to the operator's ears or side of their face. According to another embodiment, this second width can be less than the first width, wherein the second width is configured to enable air movement about the operator's face. This second width can be designed so as to leave a portion of the operator's ear uncovered so that the operator's hearing is not impaired. It can be appreciated that the welding hood can be molded so that the sides are of equal width, but extended when compared with the prior art, and to include scoring or the like or to be formed from a material that enables one to remove a portion of the side so as to facilitate air flow and uncover a portion of the operator's ear, as desired.

According to one embodiment, the first side portion can comprise a removable attachment portion that can be attached to either a right side or a left side of the face protective shield, depending upon whether the operator is right-handed or left-handed. This flexible attachment portion can be applied to either side of the welding hood, can be added to the already extended side portion, or can even be added to a side portion that has not been extended to provide extra protection. This removable attachment portion enables customization of the welding hood for the operator and provides the operator with additional protection closest to a welding area. According to a further embodiment, the first side portion can be formed from a flexible material. For additional protection, the welding hood can include a flexible attachment portion extending from a bottom portion of the face protective shield to provide protection of the operator's neck. The flexible attachment portions for the side and/or bottom of the shield can be formed from any well-known heat resistant material, such as leather, rubber, heat-resistant plastic, and the like.

In accordance with another aspect, the present disclosure is directed to a welding hood for protecting an operator's face comprising a face protective shield having a concave rearward side for receiving the operator's face, a viewing opening extending through the shield, wherein the viewing opening is located in a position in front of the eyes of the operator while the hood is fixed to the operator's head, a lens located within the viewing opening for protecting the operator's eyes while allowing the operator to look through the viewing opening, and at least one strap for securing the welding hood to the operator's head. The face protective shield can include a first side portion having a first width configured for extending over at least a portion of or completely over the ear of the operator and a second side portion having a second width. According to one embodiment, this second width can be the same as the first width so as to provide added coverage to the operator's ears or side of their face. According to another embodiment, this second width can be less than the first width, wherein the second width is configured to enable air movement about the operator's face. This second width can be designed so as to leave a portion of the operator's ear uncovered so that the operator's hearing is not impaired. It can be appreciated that the welding hood can be molded so that the sides are of equal width, but extended when compared with the prior art, and to include scoring or the like or to be formed from a material that enables one to remove a portion of the side so as to facilitate air flow and uncover a portion of the operator's ear, as desired.

According to one embodiment, the first side portion can comprise a removable attachment portion that can be attached to either a right side or a left side of the face protective shield, depending upon whether the operator is right-handed or left-handed. This flexible attachment portion can be applied to either side of the welding hood, can be added to the already extended side portion, or can even be added to a side portion that has not been extended to provide extra protection. This removable attachment portion enables customization of the welding hood for the operator and provides the operator with additional protection closest to a welding area. According to a further embodiment, the first side portion can be formed from a flexible material. For additional protection, the welding hood can include a flexible attachment portion extending from a bottom portion of the face protective shield to provide protection of the operator's neck. The flexible attachment portions for the side and/or bottom of the shield can be formed from any well-known heat resistant material, such as leather, rubber, heat-resistant plastic, and the like.

The welding hood can further include an eye box located adjacent the viewing opening. The eye box can have at least one wall framing at least a portion of the viewing opening that extends inwardly from the rearward side of the shield. According to one embodiment, the at least one wall can comprise a series of walls that completely frame the viewing opening. The walls can be configured for blocking at least one of reflective arc flashes, sunlight, and shop-lighting from entering the operator's field of view during welding. The walls of the eye box can be contoured or formed from flexible material so that they fit flush about the operator's face to reduce the amount of sparks, dirt, and/or debris from coming into contact with the operator's eyes.

The lens can be located within a holding member capable of pivoting between an open and closed position about the viewing opening. This lens can be removable from the holding member for replacement thereof.

In accordance with yet another aspect, the present disclosure is directed to a welding hood for protecting an operator's face comprising a face protective shield having a rearward side configured for receiving the operator's face and a first side portion having a first width configured for extending over at least a portion of or completely over the ear of the operator and a second side portion having a second width. The welding hood further includes a viewing opening extending through the shield, wherein the viewing opening is located in a position in front of the eyes of the operator while the hood is fixed to the operator's head. An eye box is located adjacent the viewing opening. The eye box has at least one wall, such as a series of walls, framing at least a portion or completely surrounding the viewing opening and extending inwardly from the rearward side of the shield. These walls are configured for blocking at least one of reflective arc flashes, sunlight, and shop-lighting from entering the operator's field of view during welding. A lens is located within the viewing opening for protecting the operator's eyes while allowing the operator to look through the viewing opening. The welding hood also includes at least one strap for securing the welding hood to the operator's head.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a front perspective view of a welding hood in accordance with the prior art.

FIG. 2 is a back perspective view of the welding hood of FIG. 1, in accordance with the prior art.

FIG. 3 is a side view of the welding hood of FIG. 1, positioned on the head of an operator in accordance with the prior art.

FIG. 4 is a front perspective view of the welding hood in accordance with an embodiment of the present invention.

FIG. 5 is a back perspective view of the welding hood of FIG. 4 in accordance with an embodiment of the present invention.

FIG. 6 is a top view of the welding hood of FIG. 4 in accordance with an embodiment of the present invention.

FIG. 7 is a bottom view of the welding hood of FIG. 4 in accordance with an embodiment of the present invention.

FIG. 8 is a left view of the welding hood of FIG. 4 in accordance with an embodiment of the present invention.

FIG. 9 is a right view of the welding hood of FIG. 4 in accordance with an embodiment of the present invention.

FIG. 10 is a front view of the welding hood of FIG. 4 in accordance with an embodiment of the present invention.

FIG. 11 is a back view of the welding hood of FIG. 4 in accordance with an embodiment of the present invention.

FIG. 12 is a front perspective view of a welding hood in accordance with another embodiment of the present invention.

FIG. 13 is a back perspective view of the welding hood of FIG. 12 in accordance with an embodiment of the present invention.

FIG. 14 is a top view of the welding hood of FIG. 12 in accordance with an embodiment of the present invention.

FIG. 15 is a bottom view of the welding hood of FIG. 12 in accordance with an embodiment of the present invention.

FIG. 16 is a left view of the welding hood of FIG. 12 in accordance with an embodiment of the present invention.

FIG. 17 is a right view of the welding hood of FIG. 12 in accordance with an embodiment of the present invention.

FIG. 18 is a front view of a welding hood in accordance with an embodiment of the present invention.

FIG. 19 is a side view of a welding hood in accordance with yet another embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

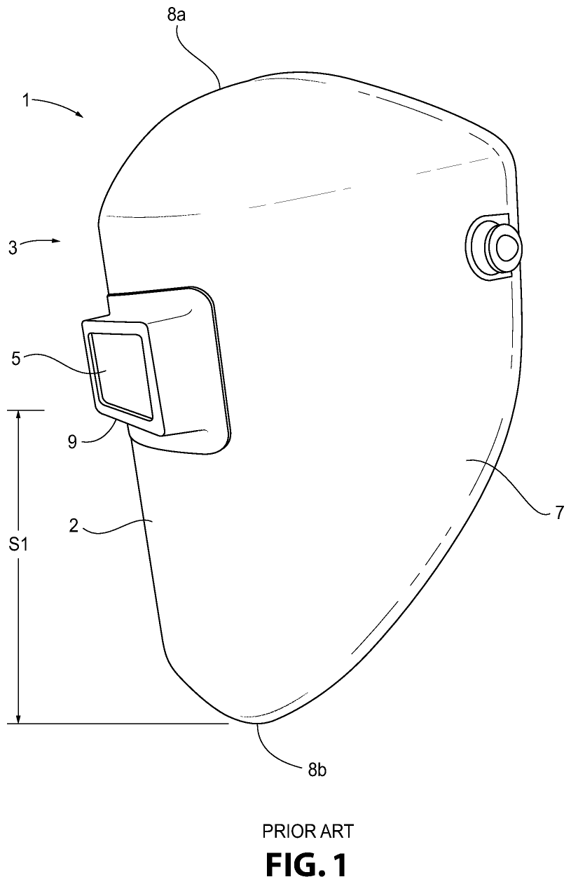

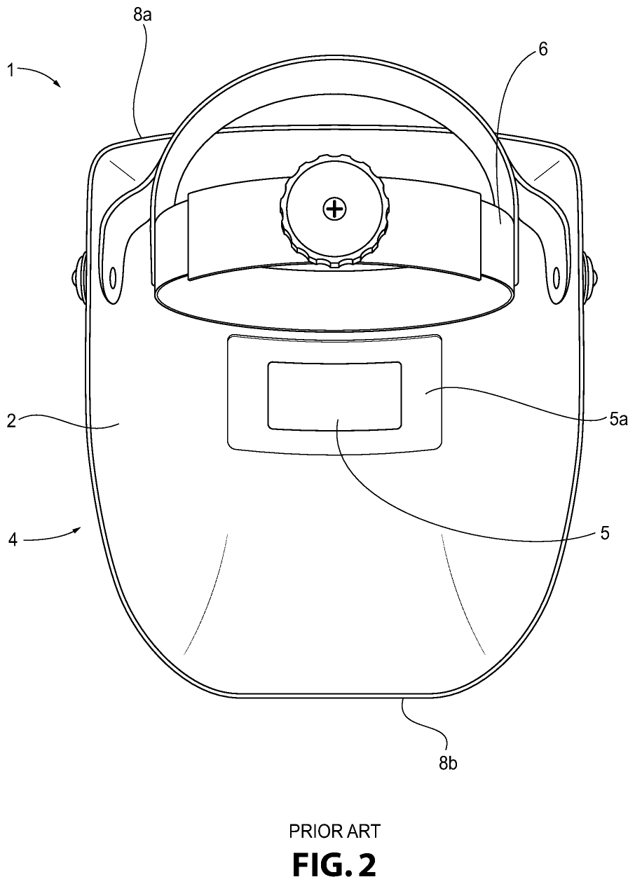

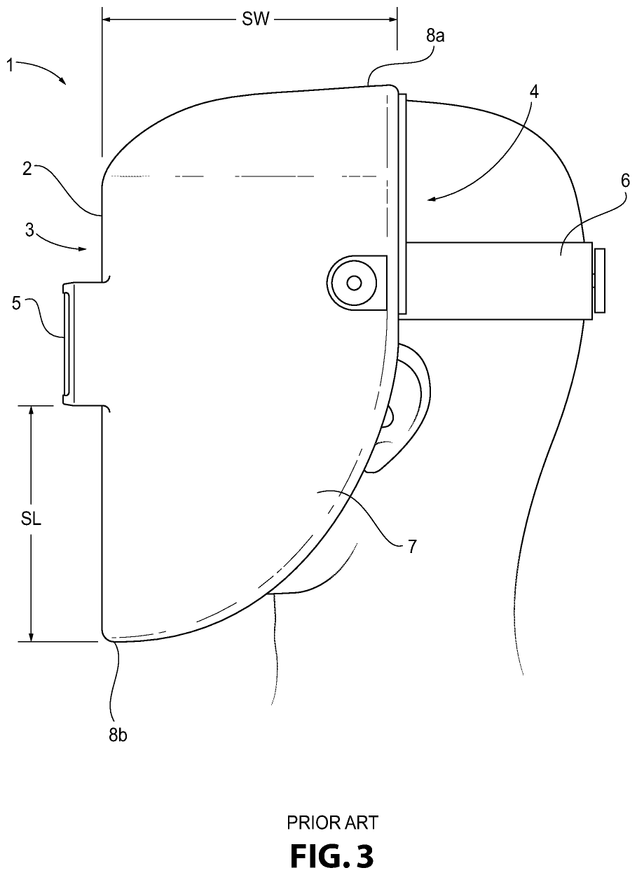

Reference is now made to FIGS. 1-3, which show one type of welding hood, generally indicated as 1, in accordance with the prior art. The welding hood 1 comprises a face protective shield 2, having a forward or front side 3, that is convex in shape, and a rearward or back side 4 that is concave in shape and configured for receiving an operator's face, a top 8a and a bottom 8b. A viewing opening or window 5 is provided so as to enable the operator to see what is being welded. A strap 6 is provided for holding the hood about the operator's face. As can be seen from FIG. 2, the area or frame 5a surrounding the viewing opening 5 is either flush with or extends slightly inward with respect to the rearward side 4 of the shield 5. As shown in FIG. 3, the welding hood 1 includes side portions 7 that only partially cover the operator's ears. Also, as can be seen in FIGS. 1-3 the bottom 8b of the hood 1 or face shield 2, does not extend very far down, leaving a significant portion of the operator's neck exposed.

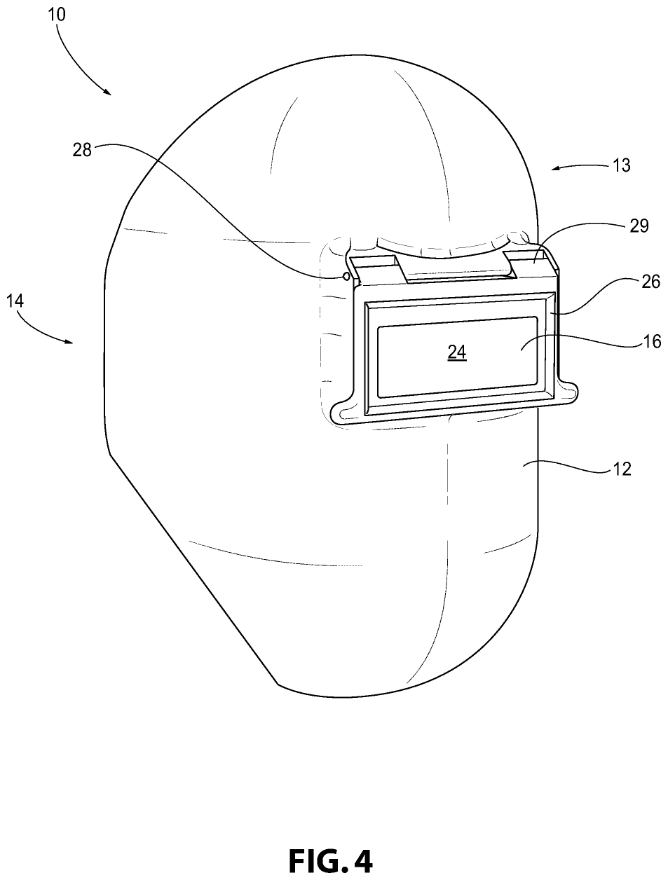

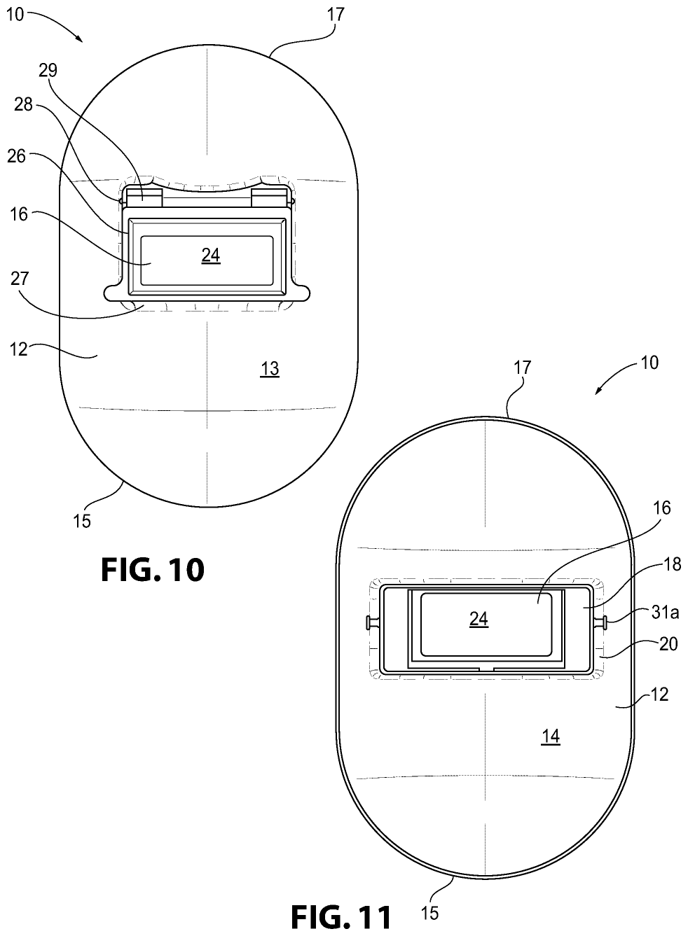

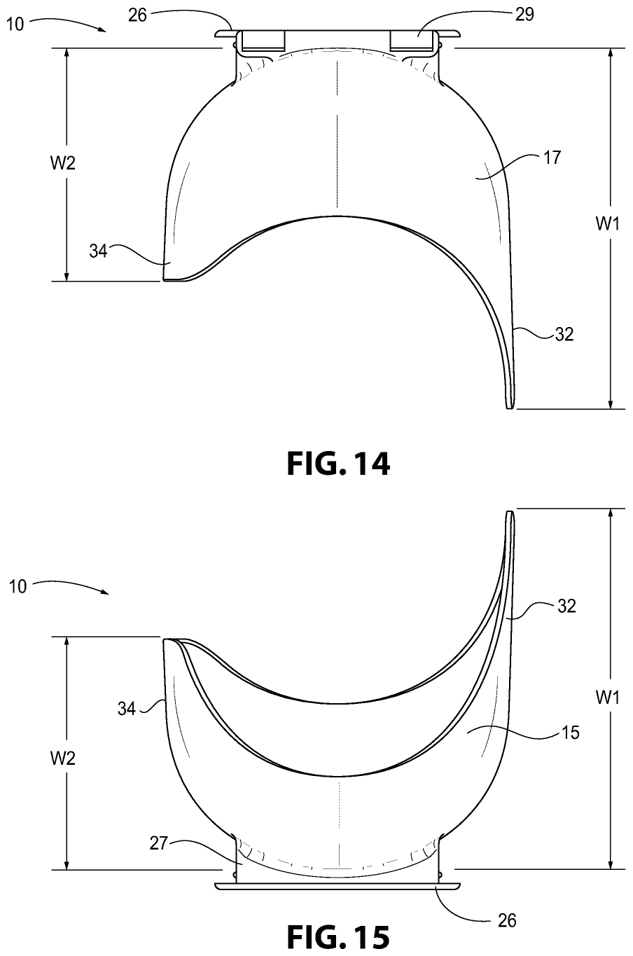

Reference is now made to FIGS. 4-19, which are directed to the welding hood, generally indicated as 10, for protecting an operator's face, as well as the operator's ears and neck, in accordance with the present disclosure. The welding hood 10 comprises a face protective shield 12 having a forward or front side 13, which is typically convex in shape, a rearward or back side 14, which is typically concave in shape, and configured for receiving the operator's face, a bottom portion 15, and a top portion 17. A viewing opening 16 extends through the face shield 12. The viewing opening 16 is located in a position in front of the eyes of the operator when the hood 10 is fixed to the operator's head. It can be appreciated that the opening 16 can have any shape, including, but not limited to, a square, rectangle, oval, a pair of circles, and the like. The welding hood 10 also includes an eye box 18, extending from the rearward or back side 14 of the hood 10, as shown in FIGS. 5, 11, and 13. The eye box 18 is located adjacent the viewing opening 16. It can also be appreciated that the eye box 18 can have any shape, including, but not limited to, a square, rectangle, oval, a pair of circles, and the like.

With continuing reference to FIGS. 5, 11, and 13, the eye box 18 has at least one wall 20 framing at least a portion of the viewing opening 16. The at least one wall 20 extends inwardly from the rearward side 14 of the shield. According to one embodiment, a plurality of walls 20 are provided for fully framing or completely surrounding the viewing opening 16 so as to block at least one of reflective arc flashes, sunlight, shop-lighting, and the like, from entering the operator's field of view during welding. The welding hood 10 also includes a lens 24 located within the viewing opening 16 for protecting the operator's eyes while allowing the operator to look through the viewing opening 16.

Still referring to FIGS. 5, 11, and 13, the walls 20 of the eye box 18 can be contoured to fit to the operator's face and surround the operator's eyes. In particular, the walls 20 can be contoured or can be formed from a flexible material, such as rubber or a foam material, so that the walls 20 fit flush against the operator's face to reduce the amount of sparks, dirt, debris and the like, from coming into contact with the operator's eyes. It can be appreciated that the walls 20 can be formed from an inflexible or rigid material, such as wood, plastic, or stiff rubber material, wherein the rigid material is shaped or contoured to fit an operator's face. It can also be appreciated that the walls 20 can be formed from a partially flexible material, such as a rubber material or a heat resistant foam rubber material, that is sufficiently flexible to conform to the operator's face, but rigid enough to provide blockage against arc flashes, light, sparks, dirt, debris, and the like.

The lens 24 can comprise a light-filtering lens for filtering light passing through the opening toward the rearward side 14 of the hood 10. The welding hood 10 can include a holding member 26 for removably holding the lens 24. The holding member 26 includes a pivoting portion 29 that is capable of pivoting via one or more pivot screws 28, between an open and closed position about the viewing opening 16. The provision of a pivoting holding member 26 enables the operator to lift the holding member 26 and lens 24 for better viewing of a fit-up or grind, if needed, or for communication with another person, without having to lift the protective shield 12 from their face. This provides an added safety measure to the welding hood 10. The holding member 26 can be configured for removably holding the lens 24 so that the lens can readily be removed and replaced with another light-filtering lens, whether for replacement due to damage or for replacement with a darker lens or with a clear lens.

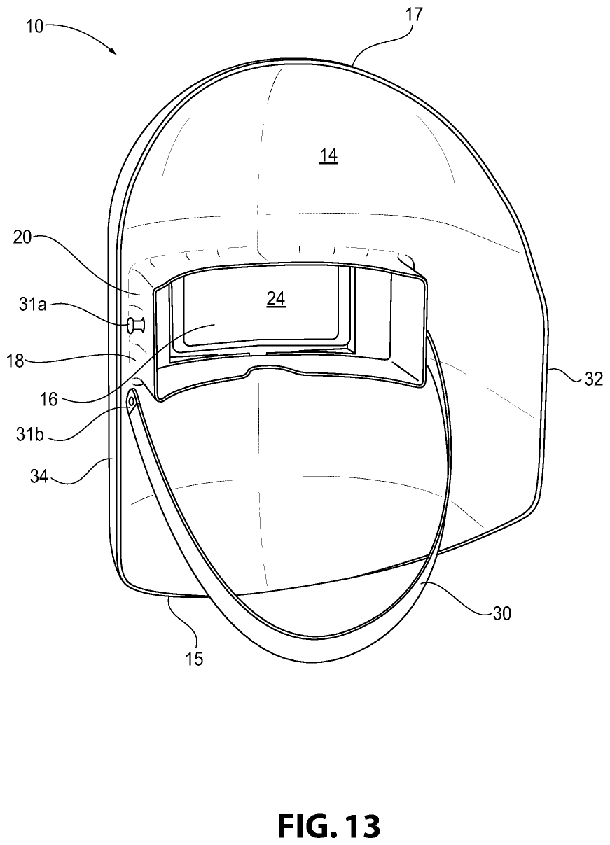

As shown in FIGS. 5 and 13, at least one strap 30 is provided for securing the welding hood 10 to the operator's head. The at least one strap 30 can comprise an elastic strap that wraps around the operator's head. According to one embodiment, a pair of extending members 31a, such as bolts, screws, and the like, can be provided on opposing walls 20 of the eye box 18 and the strap 30 can be secured to the extending members 31a, such as with hooks 31b located at the ends of the strap 30. This arrangement enables easy attachment and removal of the strap 30 about the operator's head. It can be appreciated that the strap 30 can be secured to the eye box by other well-known means, such as rivets, screws, and the like. The strap 30 can also be secured to the face shield 12, itself, by known attachment arrangements. The elastic strap 30 can be adjustable, such as with a double band and a movable buckle or any other well-known means to adjust the strap, so as to securely hold the hood 10 in place for both safety and comfort. According to one embodiment, the strap 30 can be formed from an elastic material, similar in size and shape to a strap used in ski goggles or scuba masks. The strap 30 is designed to hold the hood 10 in place, regardless of the position and/or movement of the operator's head. It can be appreciated that the strap can be formed from other known materials and can have other known designs.

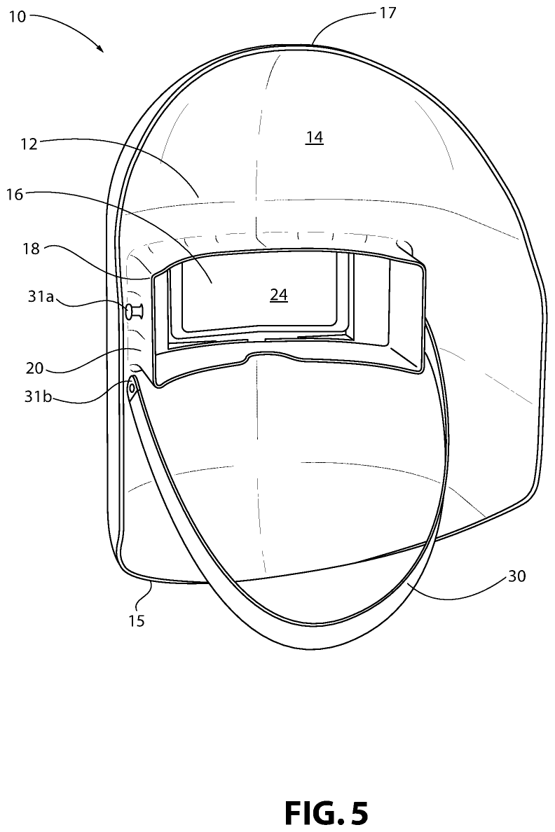

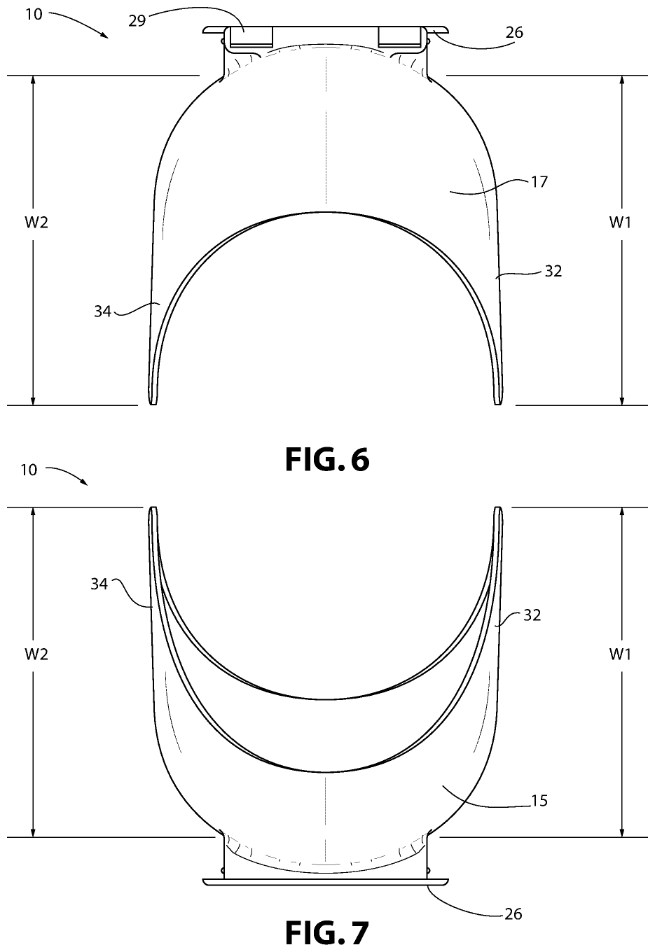

Referring now to FIGS. 6-9 and 14-17, the face protective shield 12 can include a first side portion 32 having a first width “W1” configured for extending over at least a portion of or completely covering the ear of the operator and a second side portion 34 having a second width “W2”. According to the embodiment shown in FIGS. 3-11, the first width W1 can be substantially equal to the second width “W2”. This particular design provides added coverage to the operator's ears and/or sides of their face, especially when compared with the prior art design, as illustrated in FIGS. 1-3. Prior art welding hood designs, such as shown in FIGS. 1-3, typically have a standard width “SW” from the front of the lens holder back to the rear edge on the side of about 7-7⅛ inches. As can be seen in FIG. 3, this design leaves a substantial portion of the operator's ear and/or face exposed. The welding hood of the present invention has at least one or both sides extended to a width “W1” and “W2” of approximately 8-9 inches, or 8¾ inches, from the front of the lens holder 26 back to the rear edge on the sides 32 and/or 34 so as to provide extra protection to the operator.

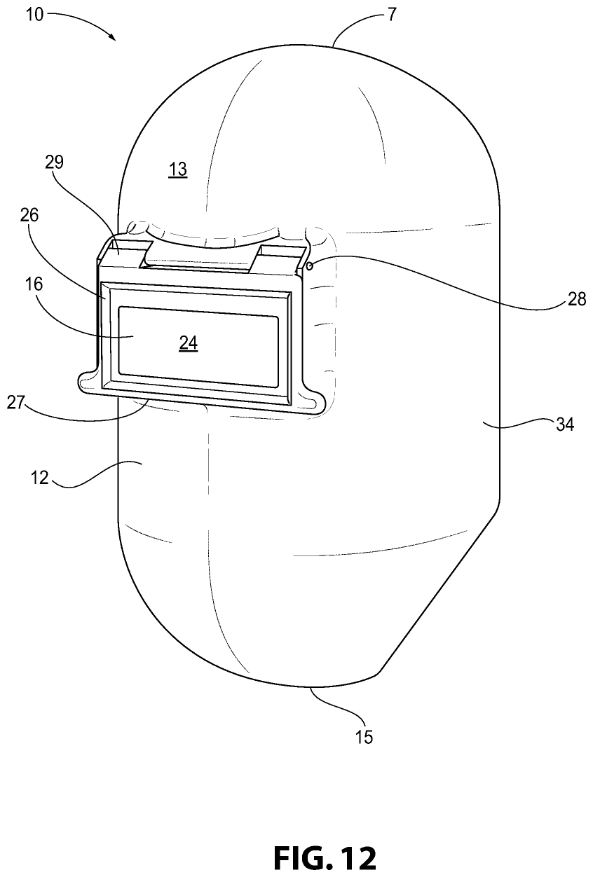

According to the embodiment shown in FIGS. 12-17, the second width “W2” can be less than the first width “W1”, wherein the second width “W2” is configured to enable air movement about the operator's face. This second width “W2” can also be designed so as to leave a portion of the operator's ear uncovered so that the operator's hearing is not impaired.

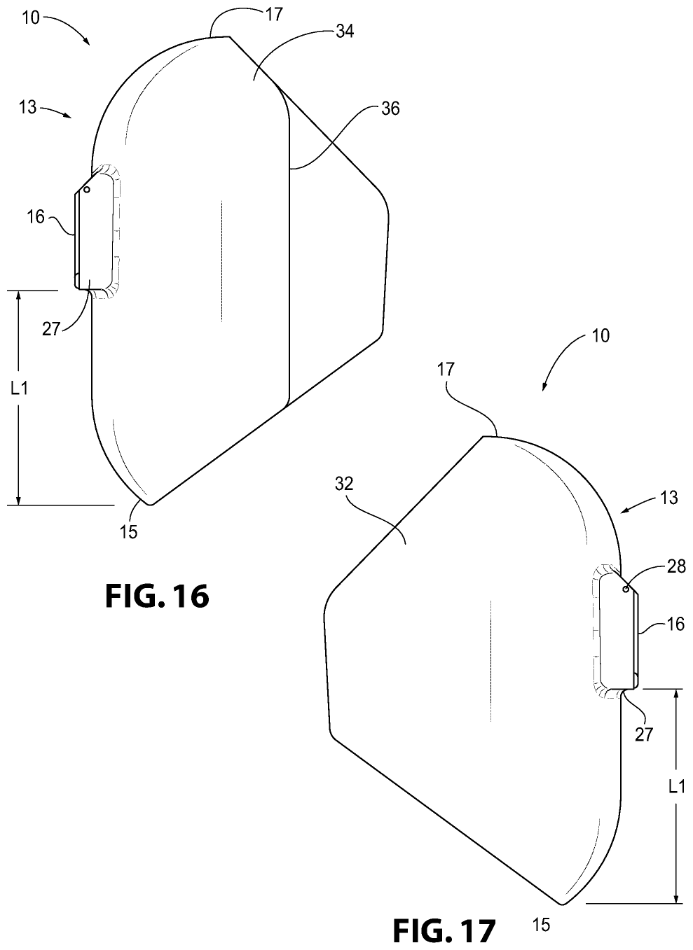

It can be appreciated that the welding hood 12 can be molded so that the first and second widths “W1” and “W2” of the first side 32 and second side 34 are of equal width, but extended when compared with the prior art. The first and second sides 32, 34 can include scoring 36, as shown in FIG. 16, or the like, to facilitate removal of a portion of one or both sides 32, 34, as needed. Alternatively, the welding hood can be formed from a material that enables one to remove a portion of one or both sides 32, 34 so as to facilitate air flow under the welding hood 10 and uncover a portion of the operator's ear and/or face, as desired.

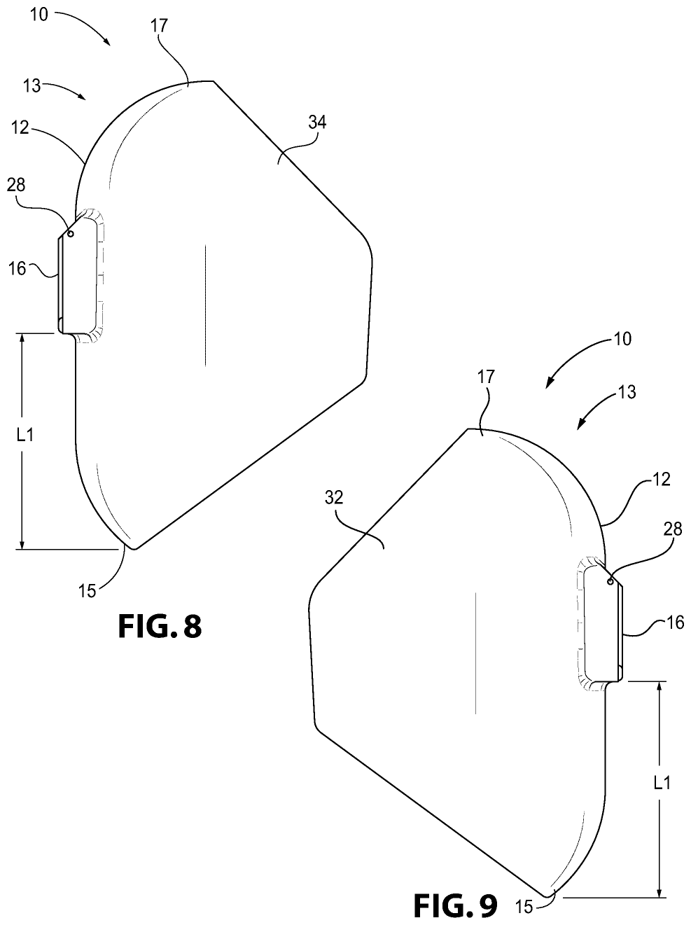

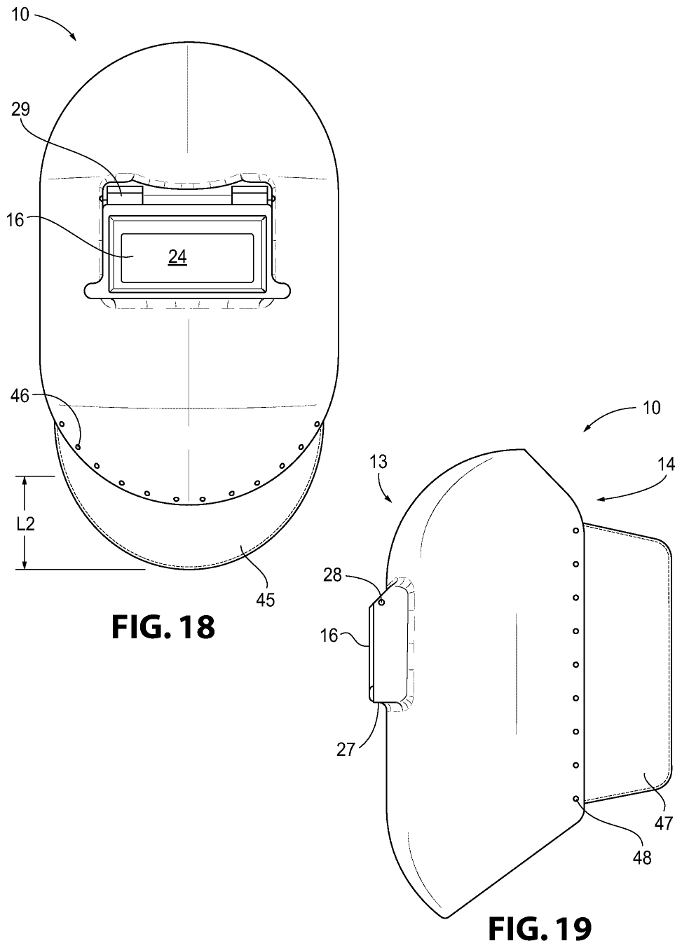

Referring back to FIGS. 1-3, the prior art hood 1 has a bottom portion 8b that extends from the bottom 9 of the viewing opening 5 for a length “S1” of approximately 7½ inches but curls under the chin, leaving a significant portion of the neck exposed, as shown in FIG. 3. In accordance with one embodiment of the present invention and with reference to FIGS. 8, 9, 16, and 17, the welding hood 10 can be designed to have a length “L1” of rigid material of approximately 6 to 7 inches or approximately 6½ inches, from the bottom 27 of the lens holding member 26, and, as shown in FIG. 18, a bottom attachment portion 45, such as leather or similar material, can be attached to the bottom portion 15 of the face shield 12 to provide additional protection of the operator's neck. This bottom attachment portion 45 can have a length “L2” of approximately 2-3 inches, or 2½ inches, and can be attached to the bottom portion 15 of the face shield 12 by any well-known means 46, such as snaps/rivets, hook and loop, auto tape, as the like, such that this bottom attachment portion drapes and/or flexes for further protection of the neck without curling under the chin. It can be appreciated that the flexible attachment portion 45 for the bottom of the face shield 12 can be formed from any well-known heat resistant material, such as leather, rubber, heat-resistant plastic, and the like. According to one example, the flexible bottom attachment portion 45 can be a three-by-twelve-inch suede split or similar material attached to the bottom portion 15 of the hood.

With reference to FIG. 19, according to one embodiment, the first or second side portion 32, 34 can comprise a removable side attachment portion that can be attached to either a right side or a left side of the face protective shield 12, depending upon whether the operator is right-handed or left-handed. This removable attachment portion 47 can be applied to either side 32, 34 of the face protective shield 12 of the welding hood 12 or it can be applied to both sides 32, 34, if desired. It can be appreciated that the removable attachment portion 47 can be added to the already extended side portion 32, 34 to provide extra protection, or can be added to a side portion that has not been extended to provide protection as needed. This removable attachment portion 47 enables customization of the welding hood 10 for the operator and provides the operator with additional protection closest to a welding area. The removable attachment portion 47 can be formed from a flexible material, such as any well-known heat resistant material, including but not limited to leather, rubber, suede, heat-resistant plastic, and the like and can be secured by any well-known securing means 48, such as snaps, hook and loop, and the like. The securing means 48 can be provided on both sides of the face protective shield 12 so that the attachment portion 47 can be secured to either side 32, 34 of the face shield 12, enabling customization of the welding hood 10, depending upon whether the operator is a right-handed or left-handed welder and/or to provide the operator with additional protection closest to a welding area.

As can be seen from the foregoing description and drawings, the welding hood of the present invention overcomes many of the deficiencies of the prior art by providing numerous safety features for the eyes, ears, and neck of the operator while also increasing the comfort to the operator. The welding hood can also be readily customized to meet a particular need of the operator and/or for use within a particular working environment.

While the disclosure has been described as having exemplary designs, the present disclosure can be further modified within the spirit and scope of this disclosure. This application is, therefore, intended to cover any variations, uses, or adaptations of the disclosure using its general principles. Further, this application is intended to cover such departures from the present disclosure as come within known or customary practice in the art to which this disclosure pertains and which fall within the limits of the appended claims.

The invention claimed is:

1. A welding hood for protecting an operator's face comprising:
a face protective shield having a rearward side configured for receiving the operator's face;
a viewing opening extending through the shield, said viewing opening located in a position in front of the eyes of the operator while the hood is fixed to the operator's head;
an eye box located adjacent the viewing opening, said eye box having at least one wall framing at least a portion of the viewing opening and extending inwardly from the rearward side of the shield, wherein the at least one wall is configured for blocking at least one of reflective arc flashes, sunlight, and shop-lighting from entering the operator's field of view during welding;
a lens located within the viewing opening for protecting the operator's eyes while allowing the operator to look through the viewing opening; and
at least one strap for securing the welding hood to the operator's head.

2. The welding hood of claim 1, wherein the at least one wall of the eye box is contoured or formed from a flexible material to fit about the operator's face.

3. The welding hood of claim 2, wherein the at least one wall of the eye box comprises a plurality of walls that are configured to fit flush against the operator's face and surround the operator's eyes to reduce the amount of sparks, dirt, and/or debris from coming into contact with the operator's eyes.

4. The welding hood of claim 1, wherein the lens comprises a light-filtering lens for filtering light passing through the opening toward the rearward side of the hood.

5. The welding hood of claim 1, wherein the lens is located within a holding member capable of pivoting between an open and closed position about the viewing opening.

6. The welding hood of claim 1, wherein the lens is located within a holding member and wherein the holding member is configured for removably holding the lens such that the lens can be replaced.

7. The welding hood of claim 1, wherein the at least one strap comprises an elastic strap that wraps around the operator's head.

8. The welding hood of claim 1, wherein the face protective shield includes a first side portion having a first width configured for extending over at least a portion of the ear of the operator and a second side portion having a second width.

9. The welding hood of claim 8, wherein the second width of the second side portion is substantially equal to the first width of the first side portion so that both the first side portion and the second side portion extend over the ears of the operator.

10. The welding hood of claim 8, wherein the second width of the second side portion is less than the first width of the first side portion, wherein the second width is configured to enable air movement about the operator's face.

11. The welding hood of claim 8, wherein the first side portion comprises a removable attachment portion that can be attached to either a right side or a left side of the face protective shield to provide the operator with additional protection closest to a welding area.

12. The welding hood of claim 11, wherein the removable attached portion is formed from a flexible material.

13. The welding hood of claim 1, including a flexible attachment portion extending from a bottom portion of the face protective shield to provide protection of the operator's neck.

14. A welding hood for protecting an operator's face comprising:
a face protective shield having a rearward side configured for receiving the operator's face;
a first side portion having a first width configured for extending over at least a portion of the ear of the operator and a second side portion having a second width;
a viewing opening extending through the shield, said viewing opening located in a position in front of the eyes of the operator while the hood is fixed to the operator's head;
a lens located within the viewing opening for protecting the operator's eyes while allowing the operator to look through the viewing opening; and
at least one strap for securing the welding hood to the operator's head.

15. The welding hood of claim 14, wherein the second width of the second side portion is less than the first width of the first side portion, wherein the second width is configured to enable air movement about the operator's face.

16. The welding hood of claim 14, wherein at least a portion of the second side portion can be removed so that the second width of the second side portion is less than the first width of the first side portion, wherein the second width is configured to enable air movement about the operator's face.

17. The welding hood of claim 14, wherein the first side portion comprises a removable attachment portion that can be attached to either a right side or a left side of the face protective shield to provide the operator with additional protection closest to a welding area.

18. The welding hood of claim 14, including a flexible attachment portion extending from a bottom portion of the face protective shield to provide protection of the operator's neck.

19. The welding hood of claim 14, including an eye box located adjacent the viewing opening, said eye box having at least one wall framing at least a portion of the viewing opening and extending inwardly from the rearward side of the shield, wherein the at least one wall is configured for blocking at least one of reflective arc flashes, sunlight, and shop-lighting from entering the operator's field of view during welding.

20. A welding hood for protecting an operator's face comprising:
a face protective shield having a rearward side configured for receiving the operator's face;
a first side portion having a first width configured for extending over at least a portion of the ear of the operator and a second side portion having a second width;
a viewing opening extending through the shield, said viewing opening located in a position in front of the eyes of the operator while the hood is fixed to the operator's head;
an eye box located adjacent the viewing opening, said eye box having at least one wall framing at least a portion of the viewing opening and extending inwardly from the rearward side of the shield, wherein the walls are configured for blocking at least one of reflective arc flashes, sunlight, and shop-lighting from entering the operator's field of view during welding;
a lens located within the viewing opening for protecting the operator's eyes while allowing the operator to look through the viewing opening; and
at least one strap for securing the welding hood to the operator's head.
